(19) United States
(12) Patent Application Publication
(10) Pub. No.: US 2026/0033765 A1
(43) Pub. Date: Feb. 5, 2026

(54) BIOSIGNAL MEASUREMENT SYSTEM

(71) Applicant: NTT.Inc, Tokyo (JP)

(72) Inventors: Kento Watanabe, Tokyo (JP); Kenichi Matsunaga, Tokyo (JP)

(21) Appl. No.: 18/872,609

(22) PCT Filed: Jun. 9, 2022
(86) PCT No.: PCT/JP2022/023295

§ 371 Date: Dec. 6, 2024

(51) Int. Cl. A61B 5/282; A61B 5/00; A61B 5/308

(52) U.S. Cl. CPC A61B 5/282; A61B 5/0024; A61B 5/0028; A61B 5/308; A61B 5/6824; A61B 5/7203

(57) ABSTRACT

An embodiment is a biosignal measurement system including a plurality of electrode devices, each including an electrode, an amplifier configured to amplify a biopotential measured via the electrode, a quantization circuit configured to convert the biopotential amplified by the amplifier into digital data to generate biopotential information, and a wireless transmitter configured to transmit the biopotential information. The biosignal measurement system further including a biosignal generation device including a wireless receiver configured to receive the biopotential information transmitted from plurality of electrode devices, and an arithmetic circuit configured to generate a biosignal waveform using the biopotential information obtained in at least two of the plurality of electrode devices.

### CROSS-REFERENCE TO RELATED APPLICATIONS

This application is a national phase entry of PCT Application No. PCT/JP2022/023295, filed on Jun. 9, 2022, which application is hereby incorporated herein by reference.

### TECHNICAL FIELD

The present disclosure relates to a biosignal measurement system for measuring a biosignal including an electrocardiographic waveform.

### BACKGROUND

In recent years, as one of methods for health management of individuals, biosignals such as electrocardiographic waveforms are recorded over a long period of time, and characteristics and changes of the waveforms are analyzed to find out the degree of activity of autonomic nerves and signs of heart disease at an early stage. As a method of acquiring a biosignal such as an electrocardiographic waveform over a long period of time, a wearable electrode in which a bioelectrode is attached to clothing has been proposed (see, for example, Non Patent Literature 1).

In the electrocardiographic waveform, which is one of biosignals, it is necessary to measure a potential difference between electrodes disposed on both left and right sides across the heart of the body. As illustrated in FIG. 7, a wearable electrode 100 of Non Patent Literature 1 includes a biopotential measuring device 400 attached to a central portion of a body, and electrodes (200, 300) which are brought into contact with left and right waist portions by wiring on compression wear.

### CITATION LIST

### Non Patent Literature

Non Patent Literature 1: Nahoko Kasai, Takayuki Ogasawara, Hiroshi Nakashima, and Shingo Tsukada, “Development of Functional Textile “hitoe”: Wearable Electrodes for Monitoring Human Vital Signals”, Communication Society Magazine, 2017-2018, Vol. 11, No. 1, pp. 17-23, The Institute of Electronics, Information and Communication Engineers, publication date: 2017 Jun. 1

### SUMMARY

### Technical Problem

When the bioelectrode is worn on the body by wearing, time and effort for wearing may cause a sense of repulsion to the wearer, and discomfort may be given to the wearer by a sense of pressure due to wearing. As an attachment position other than the body, for example, in a case where the electrodes are attached to the limbs, since the wiring linking the left and right electrodes forms a loop like handcuffs, there is a problem that the movement of the body of the wearer is constrained and a strong constraint exists.

An object of the embodiments of the present invention is to solve the above problem, and an object is to provide a biosignal measurement system capable of naturally measuring a biosignal by eliminating discomfort of a wearer and constraint on the body at the time of wearing an electrode device.

### Solution to Problem

In order to solve the above problem, a biosignal measurement system of embodiments of the present invention includes: a plurality of electrode devices including an electrode that measures a biopotential, an amplifier circuit that amplifies the measured biopotential, a quantization circuit that converts the amplified biopotential into digital data to generate biopotential information, a wireless transmitter that transmits the biopotential information, and a power supply that supplies power to the amplifier circuit, the quantization circuit, and the wireless transmitter; and a biosignal generation device including a wireless receiver that receives the biopotential information transmitted from the wireless transmitter of the electrode device, and an arithmetic circuit that generates a biosignal waveform using the biopotential information in at least two electrode devices of the plurality of electrode devices.

### Advantageous Effects of Embodiments of the Invention

According to the present disclosure, it is possible to provide a biosignal measurement system capable of naturally measuring a biosignal by eliminating discomfort of a wearer and constraint on the body at the time of wearing an electrode device.

### BRIEF DESCRIPTION OF THE DRAWINGS

FIG. 1 is a diagram illustrating a configuration example of a biosignal measurement system according to a first embodiment of the present invention.

FIG. 2 is a diagram illustrating a conceptual diagram of a biosignal measurement system according to an embodiment of the present invention.

FIG. 3 is a diagram illustrating a configuration example of a biosignal measurement system according to a second embodiment of the present invention.

FIG. 4 is an example of a measurement circuit used in a conventional biosignal measurement system.

FIG. 5 is a diagram illustrating a configuration example of a biosignal measurement system according to a third embodiment of the present invention.

FIG. 6 is a diagram illustrating another configuration example of the biosignal measurement system according to the third embodiment of the present invention.

FIG. 7 is a configuration example of a conventional biosignal measurement system.

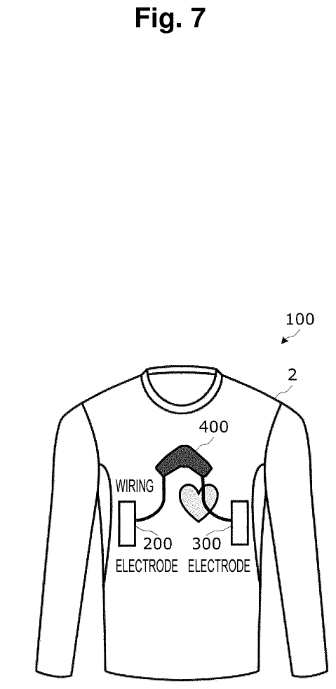

### DETAILED DESCRIPTION OF ILLUSTRATIVE EMBODIMENTS

Hereinafter, modes for carrying out embodiments of the present invention will be described with reference to the drawings. The contents of the present invention are not limited by the embodiments described below.

### First Embodiment

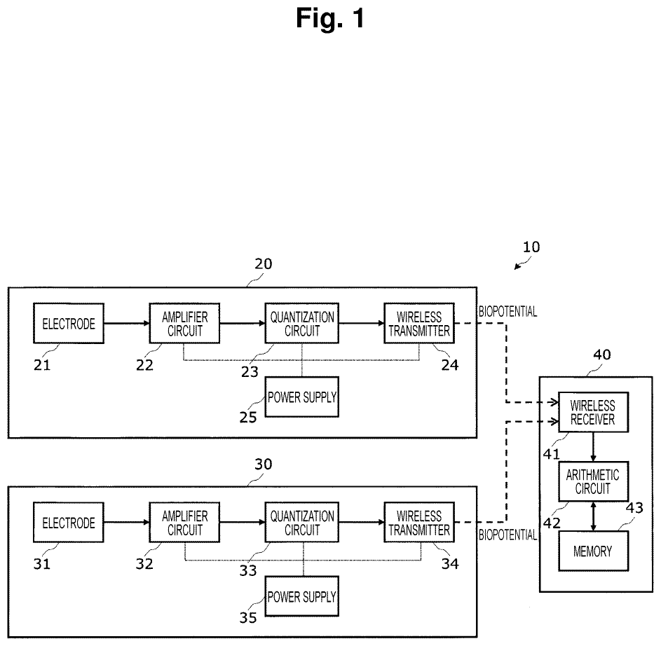

FIG. 1 is a diagram illustrating a configuration example of a biosignal measurement system according to a first embodiment of the present invention. A biosignal measurement system 10 of the present embodiment includes a plurality of electrode devices (20, 30) that measure a biopotential and a biosignal generation device 40 that generates a biosignal waveform using biopotential information in the plurality of electrode devices (20, 30).

The electrode devices (20, 30) include electrodes (21, 31) that measure a biopotential, amplifier circuits (22, 32) that amplify the measured biopotential, quantization circuits (23, 33) that convert the amplified biopotential into digital data to generate biopotential information, and wireless transmitters (24, 34) that transmit the biopotential information, and have power supplies (25, 35) that supply power to the amplifier circuits (22, 32), the quantization circuits (23, 33), and the wireless transmitters (24, 34).

The biosignal generation device 40 includes a wireless receiver 41 that receives biopotential information transmitted from the wireless transmitters (24, 34) of the electrode devices (20, 30), an arithmetic circuit 42 that generates a biosignal waveform using the biopotential information in at least two electrode devices of the plurality of electrode devices, and a memory 43 that stores the generated biosignal waveform.

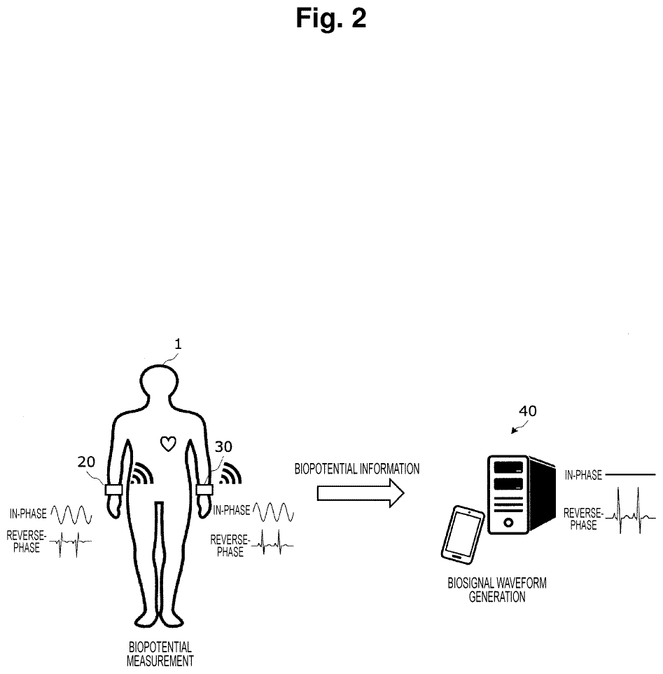

FIG. 2 illustrates a conceptual diagram of the biosignal measurement system 10 according to the present embodiment. For example, when an electrocardiogram is generated as a biosignal, it is necessary to dispose a plurality of electrode devices at positions sandwiching the heart. As an electrode device attachment form having a good sense of use for a wearer 1, for example, it is conceivable to attach the electrode device to at least two positions of the limbs such as hands and feet. By adopting such an electrode device attachment form, it is possible to greatly reduce a sense of pressure or discomfort due to wearing of wear or the like.

In each electrode device (20, 30), an in-phase component appearing as a noise component and a reverse-phase component appearing as a biopotential are measured. There is a problem that the signal of the biopotential that can be measured by the electrodes (21, 31) of the electrode devices (20, 30) is weak and the SN ratio is extremely poor.

In the biosignal measurement system 10 according to the present embodiment, signals of a plurality of measured biopotentials are transmitted to the biosignal generation device 40 by using wireless communication, and a difference operation of the signals of the biopotentials is performed by the biosignal generation device 40. By performing the difference operation, the in-phase component appearing as the noise component is removed to generate the biosignal, so that the SN ratio can be improved.

The biosignal measurement system 10 of the present embodiment is configured to transmit information of the biopotential from the plurality of electrode devices (20, 30) that measure the biopotential to the biosignal generation device that generates a biosignal waveform by using wireless communication. As a result, it is possible to provide a biosignal measurement system capable of naturally measuring a biosignal by eliminating discomfort of a wearer and constraint on the body due to physical wiring at the time of wearing the electrode device.

In FIG. 2, the case where the electrocardiogram, which is one of the biosignals, is measured has been described, but the biosignal measurement system of the present embodiment is applicable not only to the measurement of the electrocardiogram, but also to the measurement of other biosignals such as electromyograms and electroencephalogram measurement. By applying the biosignal measurement system of the present embodiment, it is possible to eliminate the discomfort of the wearer due to the physical wiring of the electrode devices, and it is possible to expect an effect of increasing the degree of freedom of electrode disposition and an increase in the range of gadgets to be mounted.

As the electrodes (21, 31) of the electrode devices (20, 30), electrodes of various materials and configurations can be used. Any electrode such as an Ag/AgCl electrode used in medical applications, a cloth electrode having conductivity, or a metal electrode can be used.

In addition, it is also possible to further improve the usability by using a non-contact electrode configuration in which an electrode is attached to the clothing by using an electrode made of cloth or metal that does not need to be directly attached to the body of the wearer.

Since the biopotential information is a very weak signal, signal amplification by the amplifier circuits (22, 32) using a filter circuit or an operational amplifier is required. In the amplifier circuits (22, 32) of the electrode devices (20, 30), a high input impedance is required in order to reduce a loss of the biopotential.

With an inverting amplifier circuit, the resistance for determining the input impedance also affects the gain setting, and further directly contributes as thermal noise, so that the SN ratio of the biopotential is lowered. On the other hand, a non-inverting amplifier circuit has a feature that noise is less likely to increase even in a high input impedance configuration. As the amplifier circuits (22, 32), it is effective to use a non-inverting amplifier circuit. By adopting the non-inverting amplifier circuit, it is possible to achieve a configuration equivalent to that of an instrumentation amplifier having a high capability of suppressing an in-phase component appearing as a noise component as a system.

Any wireless standard such as carrier communication, Wi-Fi (registered trademark), and Bluetooth (registered trademark) can be used as the wireless standard used in the wireless transmitters (24, 34) of the electrode devices (20, 30). It is sufficient if the biosignal generation device 40 that receives the information of the biopotential transmitted by the electrode devices (20, 30) is selected according to the communication standard to be used. When a short-range communication standard such as Bluetooth is used, an apparatus carried by a wearer such as a smartphone can be used, and when a short-range communication standard such as Wi-Fi is used, an apparatus such as a server can also be used.

As an electrocardiographic signal waveform that is one of biosignals, there is an electrocardiographic signal waveform called a 12-lead electrocardiographic signal waveform used for medical applications. As an electrode in the case of measuring the 12-lead electrocardiographic signal waveform, electrodes are attached to ten positions of the limbs and around the ribs of a human body, and a potential difference between a plurality of electrode pairs is measured. In the case of such an electrode disposition, since a large number of cables are entangled on the wearer's body and the wearer feels great discomfort, measurement other than in a lying position is not often performed.

By applying the biosignal measurement system of the present embodiment as a system for generating the 12-lead electrocardiographic signal waveform, all of the above-described large number of cables can be removed. As a result, it is possible to eliminate discomfort to the wearer due to a large number of cables and to achieve 12-lead electrocardiographic signal waveform measurement constantly in daily life, and it is also expected to contribute to medical progress.

### Second Embodiment

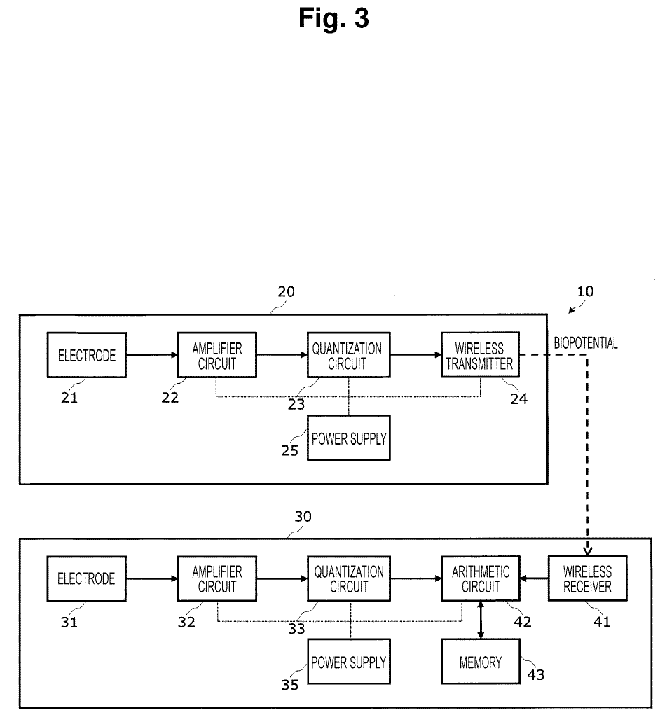

FIG. 3 is a diagram illustrating a configuration example of a biosignal measurement system according to a second embodiment of the present invention. A function required by a biosignal generation device 40 is to receive information of biopotentials transmitted from a plurality of electrode devices (20, 30) and generate a biosignal by arithmetic processing using the received information of biopotentials. As in the configuration example of FIG. 3, the function of the biosignal generation device 40 may be implemented in any of electrode devices 20, 30.

In the following description, the electrode device 30 in which the function of the biosignal generation device 40 is implemented is referred to as a master device, and the electrode device 20 that transmits a signal of the measurement potential to the master device is referred to as a slave device, and the operation of the present embodiment will be described.

A wireless receiver 41 of the master device receives the information of the biopotential measured by the slave device, and an arithmetic circuit 42 generates a biosignal using the information of the biopotential measured by the master device and the information of the biopotential measured by the slave device. The generated biosignal is stored in a memory 43 of the master device, can be used when the biosignal is analyzed, and can achieve a function similar to that of the biosignal generation device 40 of the first embodiment. In the present embodiment, since the biosignal generation device 40 is not necessary as an apparatus apart from the electrode devices, it is not necessary to carry an apparatus such as a smartphone, and it is possible to achieve biosignal measurement with less limitation on the user.

### Third Embodiment

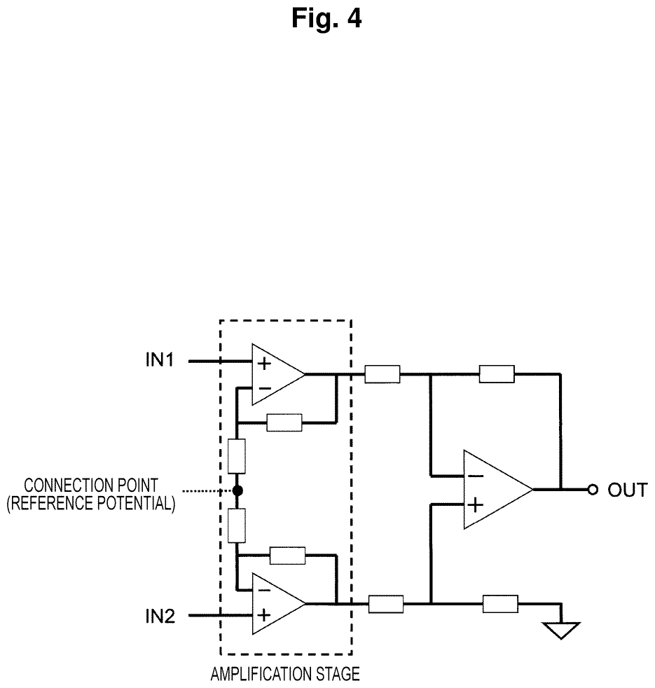

As described in the first embodiment and the second embodiment, usability is improved by using a plurality of electrode devices (20, 30) without physical wiring. On the other hand, since the electrode devices are not connected by the physical wiring, there arises a problem that the reference potentials in amplifier circuits (22, 32) of electrode devices (20, 30) do not coincide with each other. As a measurement circuit generally used in a system in which conventional physical wiring exists, there is an instrumentation amplifier as illustrated in FIG. 4.

In biosignal measurement, in order to detect a potential difference between biopotentials measured by a plurality of electrodes, it is necessary to amplify a difference between two input potentials input to an input terminal of a differential amplifier circuit at a subsequent stage of the instrumentation amplifier of FIG. 4 while greatly suppressing a noise component of an in-phase component input to the input terminal. In order to suppress the noise component of the in-phase component, it is important that inverting input terminals of two non-inverting amplifier circuits at an amplification stage preceding the instrumentation amplifier of FIG. 4 are connected to each other, that is, the reference potentials of the two non-inverting amplifier circuits are common.

The potential at the connection point of the inverting input terminals of the two non-inverting amplifier circuits converges to an average value of the two input potentials and becomes a reference potential of the two non-inverting amplifier circuits. In the first embodiment, there is no physical wiring between the electrode devices (20, 30), and the inverting input terminals of the amplifier circuits (22, 32) of the electrode devices (20, 30) are not connected by the physical wiring. Therefore, the reference potentials of the amplifier circuits (22, 32) of the respective electrode devices (20, 30) do not coincide with each other, which may deteriorate the measurement accuracy.

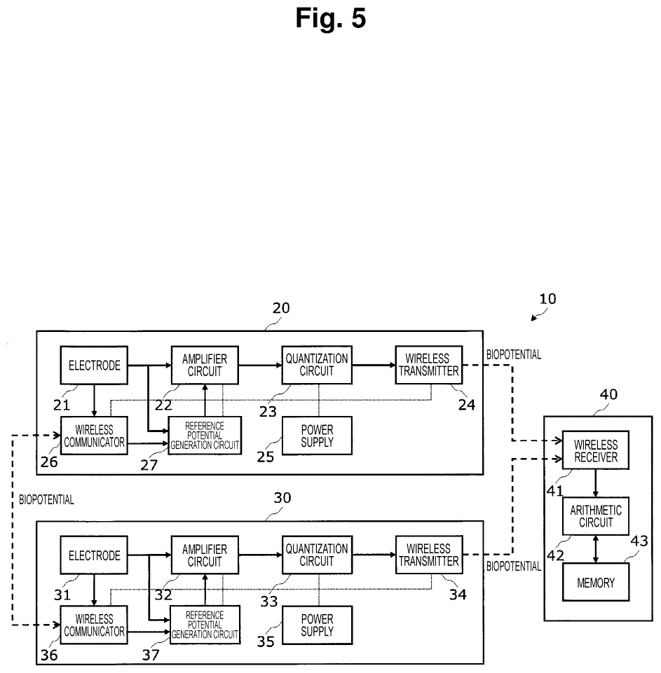

FIG. 5 is a diagram illustrating a configuration example of a biosignal measurement system according to a third embodiment of the present invention. In the third embodiment, in order to improve the biosignal measurement accuracy, the reference potentials for the amplifier circuits (22, 32) of the electrode devices (20, 30) are made common by transmitting and receiving information of the biopotential to and from the other electrode device.

In the configuration example of FIG. 5, a common reference potential generated by reference potential generation circuits (27, 37) provided in the electrode devices (20, 30) is used. As a result, signal amplification in which the reference potentials of the amplifier circuits (22, 32) are made common among the plurality of electrode devices (20, 30) becomes possible, the biosignal measurement accuracy is improved, and a good biosignal is finally obtained.

The electrode devices (20, 30) of the present embodiment include wireless communicators (26, 36) for transmitting and receiving information of a biopotential to and from the other electrode device, and the reference potential generation circuits (27, 37) that generate reference potentials of the amplifier circuits (22, 32) by using information of a biopotential (first biopotential) measured by the own electrode device and information of a biopotential (second biopotential) measured by the other electrode device. For example, in the reference potential generation circuits (27, 37), it is sufficient if the reference potentials are generated by using addition-averaging of the biopotential measured by the own electrode device and the biopotential received from the other electrode device.

In the present embodiment, the information of the biopotentials for generating the reference potential are transmitted and received by using wireless communication, so that the reference potentials of the amplifier circuits (22, 32) in the electrode devices (20, 30) can be made common without physical connection. As a result, it is possible to improve the S/N ratio of the biosignal and improve the biosignal measurement accuracy while eliminating discomfort of the wearer and constraint on the body due to physical wiring at the time of wearing the electrode device.

Since a module for wireless communication is widely distributed, it can be easily implemented at low cost. For example, it is sufficient if a modulation circuit and an antenna are provided as a transmission-side circuit of the wireless communicators (26, 36), and a demodulation circuit and an antenna are provided as a reception-side circuit. In addition, in the electrode devices (20, 30), the carrier frequencies of radio waves to be transmitted are set to frequencies different from each other, so that it is possible to transmit and receive the information of the biopotentials without interference.

### OTHER EMBODIMENTS

Optical communication may be used as another method of transmitting and receiving the biopotentials between the electrode devices. By using optical communication, it is possible to reduce the effect of widely used existing wireless communication and to stably transmit and receive signals, and it is possible to expect improvement in security due to difficulty in communication interception.

The method using optical communication can be implemented by including a modulator and an E/O converter as a transmission-side circuit of a communicator, and including an O/E converter and a demodulator as a reception-side circuit. As in the case of using wireless communication using radio waves, by setting the wavelengths of light used in the electrode devices (20, 30) to be different, it is possible to transmit and receive the biopotentials without interference.

Magnetic communication used in wireless earphones or the like is also suitable for the present embodiment. In magnetic communication, signal transmission is performed by mutual induction with another device on the basis of a magnetic field change caused by flowing a current through a coil. Since the magnetic field exhibits transmittivity to human body components such as moisture and can perform communication with low interference, it is possible to stably transmit and receive biopotential information even in the case of wearing on the human body.

In order to implement magnetic communication, it is sufficient if a modulator, a voltage-current converter represented by a transconductance amplifier, and the like, and a coil serving as an antenna are provided as the transmission-side circuit of the communicator, a coil, a current-voltage converter such as a transimpedance amplifier, and a demodulator are provided as the reception circuit.

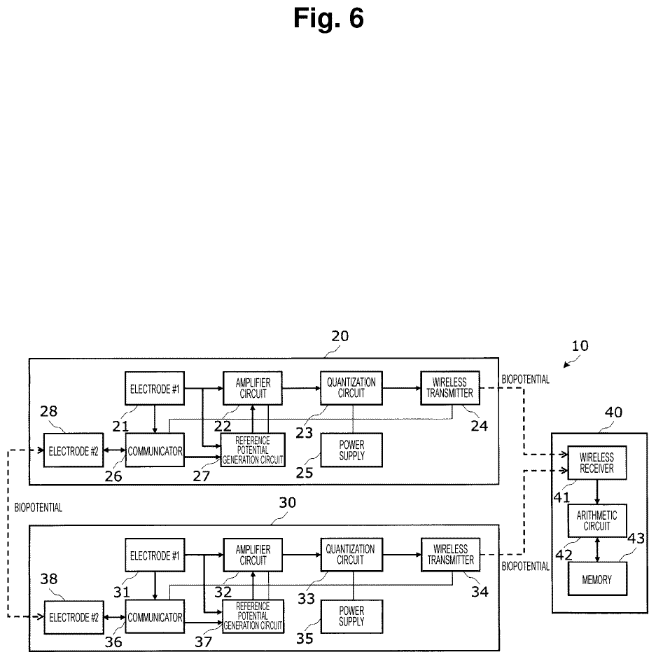

As another method of transmitting and receiving the biopotential in the electrode devices (20, 30), human body communication using a human body as a transmission path may be used. In a case where human body communication is used, as illustrated in FIG. 6, the electrode devices (20, 30) include electrodes #2 (28, 38) (second electrode) for performing human body communication in addition to electrodes #1 (21, 31) (first electrode) for measuring a biopotential.

Power for communication accounts for much of the power consumption of the electrode devices (20, 30). In spatial propagation using radio waves, the signal intensity attenuates in inverse proportion to the square of the propagation distance. On the other hand, in the case of performance via the human body, the attenuation is only inversely proportional to the propagation distance, so that transmission with less transmission power can be performed by using human body communication. Transmission and reception of the biopotential information via the human body can contribute to reduction in power consumption.

The communicators (26, 36) in FIG. 6 digitally modulate a carrier signal by using a signal obtained by sampling a biosignal provided from the electrodes #1 (21, 31), and transmits the signal from the electrodes #2 (28, 38) for human body communication to a human body, which is a transmission path. In addition, the digitally modulated biosignal transmitted from the other electrode device is received from the electrodes #2 (28, 38) for human body communication and demodulated, and the demodulated biosignal is provided to the reference potential generation circuits (27, 37).

In the reference potential generation circuits (27, 37) of FIG. 6, a common reference potential can be generated in the electrode devices (20, 30) by performing addition-averaging on the biopotential measured by the own electrode device and the biopotential of the other electrode device obtained via the human body.

By using the human body communication for transmission and reception of the information of the biopotential, it is possible to independently set a sampling rate of a biopotential signal for generating a reference potential and a sampling rate of a biopotential signal for generating a biosignal in addition to reduction in power, and there is an advantage that the degree of freedom in design is improved.

When the human body is used as the transmission path, interference can be prevented by setting the carrier frequencies used in the electrode devices to different frequencies. By setting the frequency band to be used to about several MHz to 100 MHz based on the electrical properties of the human body, the human body communication with less loss can be achieved.

As the electrode portions of the electrode devices (20, 30), an electrode for measuring the biopotential, an electrode for transmitting the biopotential, and an electrode for receiving the biopotential may be provided. In addition, the number of electrodes can be reduced by providing band pass filters having different pass bands in one electrode. By reducing the number of electrodes, the number of parts to be brought into contact with the human body is reduced, so that there is an effect of improving the comfort of the wearer.

When the carrier signals are digitally modulated in the electrode devices (20, 30), multi-level modulation such as QPSK is used, and different signal points used by the electrode devices (20, 30) are set, so that it is possible to separate signals to be transmitted and received in one carrier signal. As a result, since a common device can be used as a device used for digital modulation, mass productivity and maintainability of the device can be improved.

### INDUSTRIAL APPLICABILITY

The embodiments of the present invention can be used in a bioelectrode used for acquiring a biosignal such as an electrocardiographic signal on a daily basis and a biosignal measurement system using the bioelectrode.

### REFERENCE SIGNS LIST

1 Wearer
2 Clothing
10 Biosignal measurement system
20,30 Electrode device
21, 31 Electrode
22,32 Amplifier circuit
23,33 Quantization circuit
24,34 Wireless transmitter
25,35 Power supply
40 Biosignal generation device
41 Wireless receiver
42 Arithmetic circuit
43 Memory

1-8. (canceled)

9. A biosignal measurement system comprising:
a plurality of electrode devices, each including an electrode, an amplifier configured to amplify a biopotential measured via the electrode, a quantization circuit configured to convert the biopotential amplified by the amplifier into digital data to generate biopotential information, and a wireless transmitter configured to transmit the biopotential information; and
a biosignal generation device including a wireless receiver configured to receive the biopotential information transmitted from plurality of electrode devices, and an arithmetic circuit configured to generate a biosignal waveform using the biopotential information obtained in at least two of the plurality of electrode devices.

10. The biosignal measurement system according to claim 9, wherein
the plurality of electrode devices include a master electrode device which is one of the plurality of electrode devices and a slave electrode device which is the electrode device other than the master electrode device,
the biosignal generation device is implemented in the master electrode device, wherein the arithmetic circuit implemented in the master electrode device generates the biosignal waveform using the biopotential information obtained in the master electrode device and the biopotential information obtained in the slave electrode device.

11. The biosignal measurement system according to claim 9, wherein
the arithmetic circuit is configured to generate an electrocardiographic signal waveform by using the biopotential information transmitted from the plurality of electrode devices attached to at least two positions of limbs of a human body.

12. The biosignal measurement system according to claim 11, wherein
the arithmetic circuit is configured to generate a 12-lead electrocardiographic signal waveform by using the biopotential information transmitted from the plurality of electrode devices attached to ten positions of the limbs of the human body.

13. The biosignal measurement system according to claim 9, wherein
each of the plurality of electrode devices further includes a communicator configured to transmit a first biopotential information obtained in the electrode device to another one of the plurality of electrode devices and receive a second biopotential information obtained in the other one of the plurality of electrode devices, and
a reference potential generation circuit configured to generate a reference potential for the amplifier using the first biopotential information and the second biopotential information.

14. The biosignal measurement system according to claim 13, wherein the communicator transmits the first biopotential information via light and receives the second biopotential information via light.

15. The biosignal measurement system according to claim 13, wherein the communicator transmits the first biopotential information via magnetism and receives the second biopotential information via magnetism.

16. The biosignal measurement system according to claim 13, wherein each of the plurality of electrode devices further includes a second electrode for performing human body communication, wherein the communicator transmits the first biopotential information via a human body and receives the second biopotential information via the human body.

17. The biosignal measurement system according to claim 13, wherein the communicator transmits the first biopotential information via a radio wave and receives the second biopotential information via a radio wave.

18. An electrode device comprising:
an electrode,
an amplifier configured to amplify a biopotential measured via the electrode,
a quantization circuit configured to convert the biopotential amplified by the amplifier into digital data to generate first biopotential information,
a wireless receiver configured to receive second biopotential information transmitted from another electrode device, and
an arithmetic circuit configured to generate a biosignal waveform using the first biopotential information and the second biopotential information.

19. The electrode device according to claim 18, further comprising:
a communicator configured to transmit a first biopotential information generated by the arithmetic circuit and receive a second biopotential information obtained in another electrode device, and
a reference potential generation circuit configured to generate a reference potential for the amplifier using the first biopotential information and the second biopotential information.

20. The electrode device according to claim 19, wherein the communicator transmits the first biopotential information via light and receives the second biopotential information via light.

21. The electrode device according to claim 19, wherein the communicator transmits the first biopotential information via magnetism and receives the second biopotential information via magnetism.

22. The electrode device according to claim 19, further comprising a second electrode for performing human body communication, wherein the communicator transmits the first biopotential information via a human body and receives the second biopotential information via the human body.

23. The electrode device according to claim 19, wherein the communicator transmits the first biopotential information via a radio wave and receives the second biopotential information via a radio wave.

24. A method for generating a biosignal waveform, the method comprising:
measuring biopotentials using electrodes of a plurality of electrode devices;
amplifying the measured biopotentials in each of the plurality of electrode devices;
converting the amplified biopotentials into digital data to generate biopotential information in each of the plurality of electrode devices;
wirelessly transmitting the biopotential information from each of the plurality of electrode devices;
receiving the transmitted biopotential information from the plurality of electrode devices at a biosignal generation device; and
generating a biosignal waveform using the biopotential information obtained from at least two of the plurality of electrode devices.

25. The method according to claim 24, wherein generating the biosignal waveform comprises:
performing a difference operation on the biopotential information from at least two of the plurality of electrode devices to remove in-phase noise components.

26. The method according to claim 24, wherein the biosignal waveform is an electrocardiographic signal waveform, and wherein the plurality of electrode devices are attached to at least two positions of limbs of a human body.

27. The method according to claim 26, wherein the electrocardiographic signal waveform is a 12-lead electrocardiographic signal waveform, and wherein the plurality of electrode devices are attached to ten positions of the limbs of the human body.

28. The method according to claim 24, further comprising:
transmitting first biopotential information obtained in one of the plurality of electrode devices to another one of the plurality of electrode devices;
receiving second biopotential information obtained in the other one of the plurality of electrode devices; and
generating a reference potential for amplifying the measured biopotentials using the first biopotential information and the second biopotential information.
